(19) United States
(12) Patent Application Publication
(10) Pub. No.: US 2025/0113841 A1
(43) Pub. Date: Apr. 10, 2025

(54) Use of Black Solider Fly Larvae as Palatant / Palatability Enhancer for use in edible pet products

(71) Applicant: NPIC, Plano, TX (US)

(72) Inventors: Lingxiang Sun, Plano, TX (US); Steven Ho, Plano, TX (US); Xiaoming Liu, Plano, TX (US); Ricardo Martinez, Providence Village, TX (US); Yin-Hao Chen, Dallas, TX (US)

(21) Appl. No.: 18/482,147

(22) Filed: Oct. 6, 2023

Related U.S. Application Data
(60) Provisional application No. 63/415,209, filed on Oct. 11, 2022.

(51) Int. Cl. A23K 10/20; A23K 20/142; A23K 50/40; A61K 31/198; A61K 35/64

(52) U.S. Cl. CPC A23K 10/20; A23K 20/142; A23K 50/40; A61K 31/198; A61K 35/64

(57) ABSTRACT

The use of Black Solider Fly Larvae as Palatant/Palatability Enhancer for use in edible pet products

This invention disclosed a palatability enhancer for edible pet products using black solider fly (Hermetia illucens) larvae, wherein the black solider fly larvae are collected following the commercial farming, the greater part of the materials being processed for safety and size reduced as necessary, at 10.0%-80.0%. The palatability enhancer may also include at least one of the following: Cysteine, Arginine, Alanine, Glutamine, Aspartic acid, Glycine, Phenylalanine, Isoleucine, Histidine, Vitamin B1, and a reducing sugar, at 0.1%-10.0% by weight.

### CROSS REFERENCE TO RELATED APPLICATIONS

This application claims priority to U.S. Provisional Application No. 63/415,209 filed Oct. 11, 2022, the disclosure of which is incorporated herein by this reference.

### BACKGROUND

The pet industry is a thriving and rapidly growing sector that encompasses a wide range of businesses and services related to pets. The pet industry has witnessed consistent growth over the years. According to various reports, the global pet industry was valued at over $223 billion in 2020, and it is projected to continue growing in the coming years. The industry has shown resilience even during economic downturns, as pet owners tend to prioritize their pets' well-being.

The pet food segment holds a significant portion of the pet industry. Pet owners are increasingly concerned about the quality and nutritional value of the food they provide to their pets. Pet food were estimated to make up the largest share of the market in the United States starting from 2021. Pet food can be generally segmented into dry, wet (canned or moist), semi-moist, treats (or snacks), and raw/frozen/refrigerated dog and cat foods. Complete and balanced pet food products are formulated to be the single source of nutrition for a pet. Most pets are sustained mainly through feeding of a reduced range of commercial products and a limited number of production batches for a prolonged amount of time. This has led to a rise in demand for premium and specialized pet food options, including organic, natural, and grain-free products. The market also includes a variety of pet treats and snacks, which is another important segment of pet food, even though not necessarily so well balanced nutritionally, treats are generally more highly flavored, or have a special chewy texture, aiming at providing an enjoyable experience for the animal and its owner.

Palatability can be defined as pleasure an individual receives in association with eating a particular food, or simply how much the individual (or animal) likes the smell and taste of a certain food. Palatability in pet food refers to the taste, aroma, and overall appeal of the food to pets. It is a measure of how much a pet enjoys and is attracted to a particular food product. A highly palatable pet food is one that is appetizing and appealing to pets, encouraging them to eat and enjoy their meals. It is one of the most important aspects in edible pet products. Palatability can be improved by adding a palatability enhancer, also known as a palatant. It is specifically designed to enhance the taste and aroma of the food, making pet food more attractive to pets, encouraging them to consume an adequate amount of food and enjoy their meals. These palatants can be added in a variety of ways depending on what it is going to be applied on. For dry pet foods, palatants are usually added as a top coating while pumps of fat and liquid palatant are sprayed onto wet food. For pet treats, palatants are usually added into the treat itself or added as a topcoat after extrusion.

There are many factors that contribute to palatability acceptance in pets. The most important being smell since dogs and cats both have highly developed olfactory acuity. This is more important in cats than compared to dogs. Cats tend to be slow eaters, and tend to linger around their food longer, this allows more opportunities for their olfactive neurons to be triggered. While dogs are quick eaters, leaving a limited time for their olfactive neurons to be triggered. The second important factor is taste due to its connection to the olfactory senses. Dogs have more taste buds compared to cats, and can detect flavors such as sour, bitterness, salty, sweet, and umami. Cats on the other hand can detect similar flavors except for sweet which is completely undetectable. Factors like texture or mouth feel, in many cases can also contribute to the palatability of the food. Dogs tend to dislike larger sized kibble because they spend more time chewing. This factor is especially important for cats which are more sensitive to the size and shape of their kibble. Cats tend to reject foods with sharp edges due to its uncomfortable mouth feel.

Palatants play a crucial role in enhancing the palatability of pet food. They are specifically formulated and added to pet food to improve the taste, aroma, and overall appeal of the food to pets. Palatants are designed to enhance the taste of pet food. They add flavors that pets find appealing, such as meat, poultry, fish, or vegetable flavors. By intensifying and improving the taste of the food, palatants make it more enjoyable for pets to eat. Palatants also contribute to the aroma of pet food. They are selected and formulated to enhance the natural smells of the ingredients used in the food. The appealing aroma helps stimulate a pet's appetite and makes the food more enticing. Palatants can help mask any undesirable flavors or odors that may be present in certain ingredients or during the manufacturing process. By covering up or neutralizing these less desirable tastes, palatants make the overall food more palatable for pets. Palatants enhance the overall sensory experience of eating for pets. The improved taste and aroma make the meal more enjoyable, creating a positive association with the food and reinforcing regular and consistent eating habits.

Palatability tests are conducted by professionals such as pet food manufacturers to assess the acceptance and preferences of pets towards different food formulations. These tests help determine the palatability of a particular product and guide the development of pet foods that are more appealing to pets. Palatability tests typically involve using a group of animals as test subjects. Tests are conducted in a controlled environment to minimize external influences that may affect the results. The test subjects are usually kept in individual test chambers or cages to ensure that each animal's response is assessed independently. The consumption of each test sample is carefully measured to determine the amount of food consumed by each animal. This can be done by weighing the food before and after presentation to the animal or by using specialized feeding equipment that records intake. Test subjects may also be presented with multiple food options simultaneously to evaluate their preference rankings. This allows for a comparison of preferences among different samples or formulations. The data collected during the palatability tests are analyzed to determine the relative palatability of the samples. Statistical analysis is often performed to identify any significant differences in preference between the test groups. Palatability tests recognize that individual animals may have different preferences and responses. This is taken into account when evaluating the results, as the goal is to develop a product that appeals to a broad range of pets.

With ever-increasing needs for higher nutrients profiles, as well as realization of the importance of sustainability, insect proteins are starting to gain popularity in the pet food market. Insects, such as crickets, mealworms, and black soldier flies, are rich in protein, healthy fats, vitamins, minerals, and amino acids. They offer a complete and balanced nutritional profile, making them a valuable ingredient in pet food formulations. Insect protein is considered a sustainable protein source compared to traditional animal protein sources like beef or poultry. Insects require fewer resources, such as water, land, and feed, to produce protein. They have a low carbon footprint and can be reared using organic waste or byproducts, reducing the environmental impact of pet food production. Insect protein also offers a unique and novel protein option for pets. Introducing novel protein sources in pet food formulations can help prevent or manage food sensitivities that may arise from prolonged exposure to the same protein sources.

Among popular insect proteins, black soldier fly (Hermetia illucens) larvae, aka BSFL, are gaining the most traction for its versatility of form factors and extremely high nutrient density. The primary benefit of using black soldier fly larvae, is that it being sustainable and also environmentally friendly. Insect farming as of today is with high efficiency, requiring very little room and helping reduce greenhouse gas emissions. Black soldier fly larvae require little to reproduce during cultivation, and are fast growing, with extremely high food-to-body weight conversion rate. Black soldier fly larvae can be reared using organic waste materials, such as food scraps or agricultural byproducts. This makes them a sustainable protein source, as they can help convert waste into valuable nutrients while minimizing environmental impact. Besides, black soldier fly larvae provide a variety of different essential nutrients, containing at least 50% of protein, 35% of fats, needed for any animal or human. They are also rich in copper, iron, B vitamins, manganese, phosphorus, selenium, and zinc, making black solider fly larvae a great choice to incorporate into a pet's diet.

### SUMMARY

The described invention comprises an edible pet product palatability enhancer using black solider fly larvae at 10.0%-80.0% by weight, also with Cysteine and Arginine, at 0.1%-10.0%, and Lysine at 0.1%-10.0%, by weight that provides maximized palatability at moderate application level when used internally or topically.

In a first aspect, the invention provides a food composition that enhance the performance of edible pet products, where the use of black solider fly larvae at 10.0%-80.0% by weight, and other amino acids combined for example Cysteine, Arginine and Lysine, will provide rich and diversified aroma that significantly enhance the palatability, where the black solider fly larvae hydrolysate is either directly purchased or hydrolyzed in-house, where the proteins are hydrolyzed a certain degree where 90% under 5,000-10,000 Dalton. The amino acids profile of described hydrolysate comprises: 1.0%-10.0% of Aspartic acid, 1.0%-10.0% of Threonine, 1.0%-10.0% of Serine, 1.0%-15.0% of Glutamic Acid, 1.0%-10.0% of Glycine, 1.0%-10.0% of Alanine, 0.05%-10.0% of Cystine, 1.0%-10.0% of Valine, 0.05%-10.0% of Methionine, 0.5%-10.0% of Isoleucine, 1.0%-10.0% of Leucine, 0.5%-10.0% of Tyrosine, 1.0%-10.0% of Phenylalanine, 1.0%-10.0% of Lysine, 0.5%-10.0% of Histidine, 1.0%-10.0% of Arginine, on dry matter basis. The added amino acids will assist the enrichment of flavor profiles, where Cysteine will contribute meat/caramel types of aroma, Arginine will contribute bread/butter/caramel types of aroma, Lysine will contribute bread/caramel types of aroma. The enhancer described comprises protein at 5.0%-40.0%, fat at 0.5%-40.0%, ash at 0.01%-5.0%, moisture at 50.0%-20.0%. The preferred range of addition of described enhancer in edible pet products is about 0.5%-50.0%.

In another aspect, the invention comprises thermal processing condition of 210-300 degree Fahrenheit for 20-100 minutes, for the completion of series Maillard reactions. Vitamin B1 at 0.1%-10.0% is introduced as processing aids for the optimization of performance. Reducing sugars of Xylose and Fructose at 0.5%-10.0% each by weight are introduced for the completion of Maillard reactions as well as performance optimization. Mixed Tocopherols as antioxidant may be added to the described composition during/after the thermal process.

The described invention can be included with any additional ingredients which provide adequate nutrition, and/or animal health benefits, and/or structural strength for the forming of edible pet products. The described composition along with/without additional ingredients may be formed through injection molding processes and/or cold and hot extrusion process to form edible pet products. The described composition may also be applied topically—also referred to as Coating in pet industry, to deposit the palatability enhancer onto the surface of edible products by spraying, dusting, etc.

The described invention will significantly improve the palatability performance of edible pet products, comparing to major leading brands in the market. The invention will be described with the following examples.

### BRIEF DESCRIPTION OF THE DRAWINGS

FIG. 1 shows an embodiment wherein a comparison palatability test among products with palatant described under this patent application and a commercialized palatant Willow (6C2 AQ) 100264.

FIG. 2 shows an embodiment wherein an assessment of significance in consumption difference among products with palatant described under this patent application and product with a commercialized palatant, Willow (6C2 AQ) 100264.

### DETAILED DESCRIPTION

The acceptance palatability trials are conducted by professional animal farms registered with USDA under the Animal Welfare Act. Twenty pets (dogs/cats) with identifications are used for the test. The pets receive normal diets on regular basis and at least two hours prior to the trials. The trial duration is two days. The pets have access to the treats not to exceed thirty minutes. Amount consumed and first choice are documented for each pet. The following animal behaviors might also be documented, if needed or applicable: chewed, sniffed, licked, played with, picked up, dropped, lost interest, no interest, and time to finish.

The comparison palatability trials are conducted by professional animal farms registered with USDA under the Animal Welfare Act, using the industry standard two bowl comparison test. Twenty pets (dogs/cats) with identifications are used for the test. The pets receive normal diets on regular basis and at least two hours prior to the trials. The trial duration is two days with treat placement switched each day to avoid handedness and bias. The pets have access to the treats until they finished the equivalent of one treat, but not to exceed thirty minutes. One treat of each variable is provided to each pet each day. Amount consumed and first choice are documented for each pet. The following animal behaviors might also be documented, if needed or applicable: first approach, chewed, sniffed, licked, played with, picked up, dropped, lost interest, no interest, and time to finish. A Chi-squared test (Chi2 test) and/or a separate paired t-Test will be conducted for data analysis to determine the preference whether two groups of products are chosen equally or not.

### Example 1

The Table. 1 and Table. 2 shown here illustrate the palatability acceptance difference between the use of palatant and the use without. The carrier being a standardized basal formula, where it comprises 20.0%-50.0% rice flour, 5.0%-20.0% vegetable glycerin, 5.0%-20.0% water, by weight, and necessary amount of nutrient and processing additives. The material compositions are processed with twin screw hot extruders, ranging from 75 degree Centigrade to 140 degree Centigrade. Table 1 reflects the situation where no palatant is added. Table 2 reflects the situation where palatant is added at 0.5%-5.0% by weight.

TABLE 1
Total Treats Consumption

| | Number | Treats | Treats | |
|---|---|---|---|---|
| Duration | of Dogs | Offered | Consumed | Percentage |
| 2 DAYS | 20 | 1499 grams | 678 grams | 45.2% |

TABLE 2
Total Treats Consumption

| | Number | Treats | Treats | |
|---|---|---|---|---|
| Duration | of Dogs | Offered | Consumed | Percentage |
| 2 DAYS | 20 | 1468 grams | 1400 grams | 95.4% |

Basing on the results from acceptance palatability test, over a 2-day period the products with palatant (Table. 1) added showed a roughly twice the consumption rate comparing to the products without palatant (Table. 2) among testing dogs. It also indicated the products without palatant performed poorly, with less than 50%, yet products with palatant performed good with more than 95% acceptance rate.

### Example 2

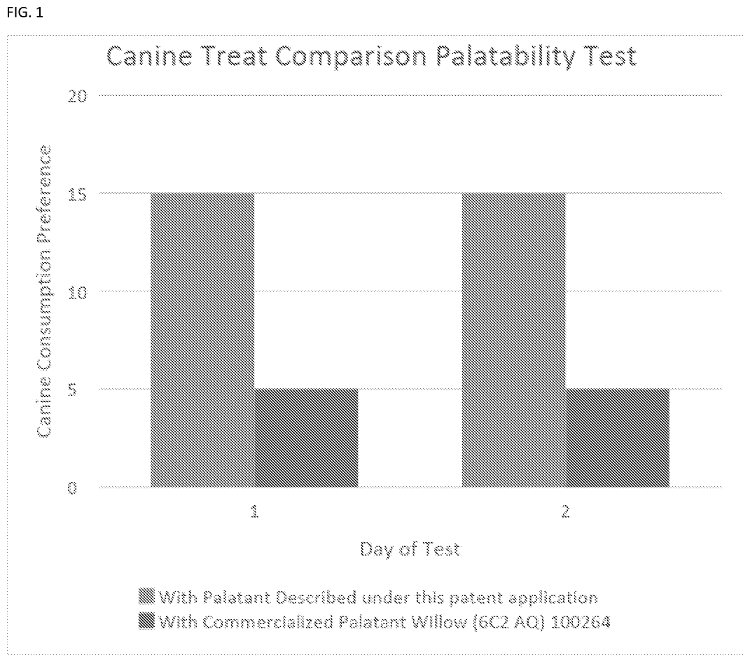

The FIG. 1 shown here illustrate the comparison palatability difference among dogs between products with the use of the palatant described under this patent application and a commercialized palatant, Willow (6C2 AQ) 100264, from company AFB International. The carrier being a standardized basal formula, where it comprises 20.0%-50.0% rice flour, 5.0%-20.0% vegetable glycerin, 5.0%-20.0% water, by weight, and necessary amount of nutrient and processing additives. The material compositions are processed with twin screw hot extruders, ranging from 75 degree Centigrade to 140 degree Centigrade.

Table. 3 reflects the summary of Chi-squared test regarding the comparison palatability test among products with palatant described under this patent application and a commercialized palatant Willow (6C2 AQ) 100264, at an inclusion ratio of 1:1, results shown in FIG. 1.

TABLE 3
Daily Preference Count

| Day | Treat A | Treat B | P of Chi² | Significant |
|---|---|---|---|---|
| 1 | 15 | 5 | 0.0253 | YES! |
| 2 | 15 | 5 | 0.0253 | YES! |

Basing on the results from comparison palatability test among dogs, the palatant described under this patent application outperforms with statistically meaningful results, comparing to product with a commercialized palatant, Willow (6C2 AQ) 100264, from company AFB International.

### Example 3

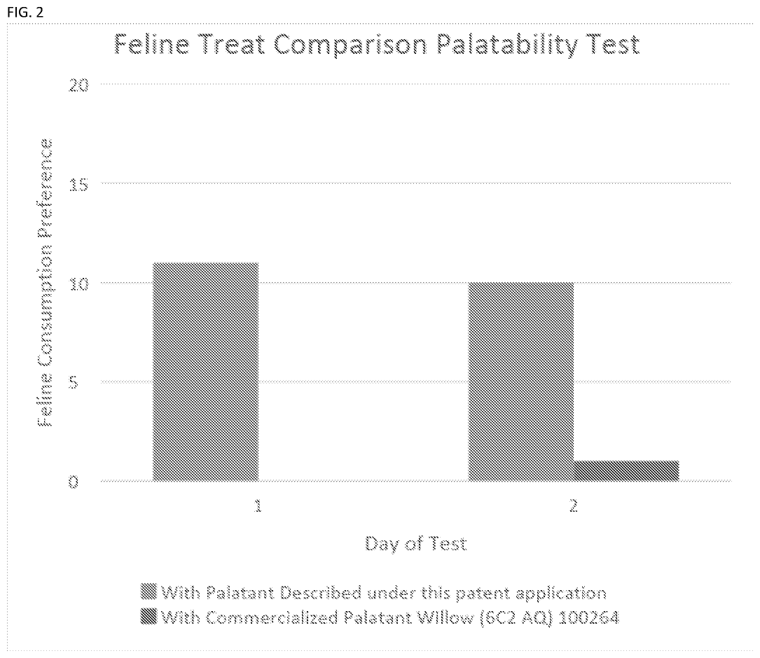

The FIG. 2 shown here illustrate the comparison palatability difference between products with the use of the palatant described under this patent application and a commercialized palatant, Willow (6C2 AQ) 100264, from company AFB International among cats. The carrier being a standardized basal formula, where it comprises 20.0%-50.0% corn starch and wheat gluten, 5.0%-20.0% vegetable glycerin, 5.0%-20.0% water, by weight, and necessary amount of nutrient and processing additives. The material compositions are processed with twin screw hot extruders, ranging from 75 degree Centigrade to 140 degree Centigrade.

Table. 4 reflects the summary separate paired t-Tests to assess significance in consumption difference among products with palatant described under this patent application and product with a commercialized palatant, Willow (6C2 AQ) 100264, from company AFB International among test cats, results shown in FIG. 2.

TABLE 4

| | Average Daily Consumption Data | | | | |
|---|---|---|---|---|---|
| Day | Treat A | Treat B | Total | P of t | Significant |
| 1 | 5.5 | 0.0 | 5.5 | 0.0004 | YES! |
| 2 | 5.4 | 0.0 | 5.4 | 0.0002 | YES! |

Basing on the results from comparison palatability test among cats, the palatant described under this patent application outperforms with statistically meaningful results, comparing to product with a commercialized palatant, Willow (6C2 AQ) 100264.

Although the use of the invention has been described in details with examples and illustrations, it is not limited to the specific material combination nor manufacturing processes disclosed hereinabove, and that many modifications, equivalents, and alternatives are intended to be included within the scope of the claims.

It should be understood that various changes and modifications to the presently preferred embodiments described herein will be apparent to those skilled in the art. Such changes and modifications can be made without departing from the spirit and scope of the present subject and without diminishing its intended advantages. It is therefore intended that such changes and modifications be covered by the appended claims.

1. A composition that increases palatability of an edible pet product, comprising
cysteine at 0.1%-10% by weight;
arginine at 0.1%-10% by weight;
black solider fly larvae at 10.0%-80.0% by weight;
one or more amino acids other than cysteine or arginine each at 0.1%-10.0% by weight; and
one or more processing aids each at 0.1%-10.0% by weight.

2. The composition of claim 1, wherein the one or more amino acids other than cysteine or arginine are selected from a group of alanine, glutamine, aspartic acid, glycine, phenylalanine, isoleucine, and histidine.

3. The composition of claim 1, wherein the one or more processing aids are selected from a group of vitamin B1, xylose, fructose, and mixed tocopherols.

4. The composition of claim 1, wherein the black solider fly larvae is hydrolyzed and breakdown to 10,000 Dalton or under.

5. A method of making a composition that increases palatability of an edible pet product comprising steps of,
making a mixture comprising cysteine at 0.1%-10% by weight; arginine at 0.1%-10% by weight;
black solider fly larvae at 10.0%-80.0% by weight; one or more amino acids other than cysteine or arginine each at 0.1%-10.0% by weight; and one or more processing aids each at 0.1%-10.0% by weight;
acidify the mixture to a pH in a range of about 2.0 and 7.0; and
treat the mixture at about 210 to 300 degree Fahrenheit for about 20 to 100 minutes.

6. The method of claim 5, wherein the one or more amino acids other than cysteine or arginine are selected from a group of alanine, glutamine, aspartic acid, glycine, phenylalanine, isoleucine, and histidine.

7. The method of claim 5, wherein the one or more processing aids are selected from a group of vitamin B1, xylose, fructose, and mixed tocopherols.

8. The method of claim 5, wherein processed material comprises protein at 5.0%-40.0%, fat at 0.5%-40.0%, ash at 0.01%-5.0%, moisture at 20.0%-80.0%.

9. The method of claim 5, wherein processed material deposits 0.5%-50.0% by weight in edible pet products.
